(19) United States
(12) Patent Application Publication
(10) Pub. No.: US 2006/0240510 A1
(43) Pub. Date: Oct. 26, 2006

(54) Method for designing peptides

(75) Inventors: Heli Valtanen, Soderkulla (FI); Mikael Björklund, Helsinki (FI); Erkki Koivunen, Helsinki (FI)

Correspondence Address: BIRCH STEWART KOLASCH & BIRCH, PO BOX 747, FALLS CHURCH, VA, 22040-0747, US

(73) Assignee: CTT Cancer Targeting Technologies OY, Helsinki (FI)

(21) Appl. No.: 10/528,989

(22) PCT Filed: Sep. 29, 2003
(86) PCT No.: PCT/FI03/00705

§ 371 Date: Oct. 25, 2005

(30) Foreign Application Priority Data
Sep. 27, 2002 (FI) 20021726

(51) Int. Cl. C07K 14/47; C12N 15/74; C07H 21/04; C12P 21/06; C12N 1/21

(52) U.S. Cl. 435/69.1; 435/472; 435/252.33; 530/350; 536/23.5

(57) ABSTRACT

The present invention relates to genetic engineering and; in specific, to design, generation, and modification of recombinant peptides using a combination of phage display and intein-mediated protein cleavage reaction.

### FIELD OF THE INVENTION

The present invention relates to genetic engineering and, in specific, to design, generation and modification of recombinant peptides using a combination of phage display and intein-mediated protein cleavage reaction.

### BACKGROUND OF THE INVENTION

Phage display and other high throughput screening methods have been used to obtain small molecular weight peptides that bind to selected receptors or other targets. Although peptides that bind to a target can be identified quite rapidly by biopanning, development of these sequences into useful high affinity peptides can take a substantial amount of time. Furthermore, combinatorial methods such as phage display usually identify peptides based on binding interaction alone and thus finding of a biologically active peptide with sufficient water solubility may require testing of several candidates. Unfortunately, the preparation of a series of different peptides by chemical synthesis becomes laborious and expensive, especially when the peptides need to be cyclized using specific disulfide bond arrangements.

To accelerate the identification of peptides with desired activity, peptides have been produced as fusion proteins with glutathione-S-transferase or alkaline phosphatase. However, a peptide may lose its activity when fused to a carrier protein. Furthermore, when the peptide is an enzyme inhibitor, a fusion protein may not be well suited for the demonstration of such an activity. These problems can be avoided by releasing the peptide from the carrier using proteases, or peptide-bond hydrolysing chemicals such as cyanogen bromide or hydroxylamine, but the peptide yield is often very low. The use of these agents may also result in the degradation of the peptide itself.

Peptides found by phage display system may also be quite insoluble in water, which makes them difficult to study and use in biological systems.

An intein-mediated protein splicing system has been described for the preparation of recombinant proteins (Chong et al., 1997). Inteins are proteins harboring protein-splicing activity and are commonly utilized as fusion partners to express recombinant proteins in bacteria. The self-cleaving ability of the intein allows the separation of the target protein from the intein so that no treatment with proteinase or peptide-bond-hydrolysing chemical is required (Chong et al., 1997; Mathys et al., 1999).

The splicing activity of inteins is inducible with e.g. thiol reagents or, on the other hand, with temperature and pH changes (Evans et al., 1999).

Intein system has been applied to producing small cyclic peptides (WO 00/36093). The methods disclosed therein utilize the trans-splicing ability of split inteins to catalyze cyclization of peptides. The peptides produced in said publication are backbone-cyclic peptides, i.e. those having a peptide bond between the N— and C-terminal amino acids. In the method a target peptide is interposed between two portions of a split intein, which structure is essential for obtaining backbone-cyclized peptides.

While phage display is a powerful tool to select novel peptide ligands, current phage display libraries have a limited chemical diversity, as they must rely on the use of the twenty naturally occurring amino acids. There have been a few in vitro and in vivo approaches to add additional amino acids into phage displayed peptides and proteins. Also synthetic peptides with unnatural amino acids have been ligated to a phage-displayed protein that has been modified via phage-display mutagenesis (Dwyer et al., 2000).

The incorporation of amino acid analogues to increase the chemical diversity of the phage display peptides is highly important as it could lead to the identification of more active and stable peptides that could better serve as lead compounds for the drug discovery process.

### SUMMARY OF THE INVENTION

We have now designed improved methods for peptide production and modification by exploiting the intein-mediated protein splicing as a method to rapidly produce phage display peptides in a soluble form. The methods of the invention are particularly useful in producing peptides having disulfide bridges, the peptide cleavage being carried out using temperature/pH-inducible intein splicing. As an example the production of the dodecapeptide inhibitor of gelatinases CTTHWGFTLC (CTT) is described. CTT is a disulfide bond-containing low molecular weight peptide that has been discovered by screening random peptide libraries displayed on filamentous phage.

The intein system also allowed us to prepare CTT peptide variants, which contain unnatural amino acids. The CTT peptide containing 5-fluorotryptophan turned out to be more stable in human serum and a more potent inhibitor of tumor cell invasion than the wild type CTT peptide.

CTT-peptide is soluble in water. However, for labelling purposes it would be necessary to insert an additional tyrosine residue in the peptide. A chemically synthesized, modified CTT peptide with such additional tyrosine was, however, insoluble in water and made the peptide impractical to be used in laboratory. Consequently, we expressed a combinatorial library of CTT peptide containing an additional tyrosine flanked by random hydrophobic amino acids as an intein fusion, and tested the resulting peptides for solubility and activity. We found that using this system, peptides with improved solubility properties can be conveniently screened.

We thus used intein-mediated protein cleavage reaction for the generation of recombinant peptides in E. coli. The method allowed rapid production and purification of the ten-residue long gelatinase inhibitor peptide CTTHWGFTLC in milligram quantities. Alanine scanning mutagenesis of the peptide showed that the tryptophan residue is central for the gelatinase inhibitory activity. Intein cleavage also occurred after biosynthetic incorporation of hydroxylated and fluorinated tryptophan analogues into the intein fusion protein. The analogues were incorporated efficiently using a protein expression strain converted to a tryptophan auxotroph by insertional mutagenesis using in vitro assembled bacteriophage Mu DNA transposition complexes. All tryptophan analogue-containing peptides retained the gelatinase inhibitory activity. 5-fluorotryptophan-containing peptide showed enhanced stability in serum and was more potent inhibitor of tumor cell invasion than the wild type CTTHWGFTLC peptide. These studies open new possibilities to modify peptides and improve their activity by biosynthetic incorporation of unnatural amino acids. Collectively these studies show that intein-mediated expression of peptides is a versatile tool for peptide design and may enable development of highly active peptides with potential therapeutic applications.

Furthermore, we performed phage selection using proMMP-9 as a target. After three rounds of selection, we cloned the resulting peptides in an intein vector using a pair of oligonucleotide primers that were designed so that any phage peptide insert can be amplified without the knowledge of the peptide sequence. The resulting peptides have a sequence ADGA-(X)ₙ-GAAG, where the ADGA and GAAG amino acid sequences are derived from the phage and (X)ₙ is the peptide insertion. As an example, two such peptides were successfully expressed and their specificity could be shown by inhibition of phage binding.

In addition, a peptide display system is described, where an auxotrophic E. coli is used for the incorporation of amino acid analogues into phage particles. This system may facilitate selection of peptides with improved activity or stability. In an auxotrophic bacterial strain the amino acid auxotrophism enforces the misaminoacylation of transfer RNAs in the absence of the naturally occurring amino acid with subsequent incorporation of the amino acid analogues into polypeptides. Said method is used herein for the production of phage particles.

Consequently, the present invention is generally directed to a method for producing a peptide, comprising the steps of providing a nucleic acid molecule encoding a polypeptide comprising a peptide of interest, incorporating said nucleic acid molecule into an expression vector as a fusion with an intein, and expressing the peptide-intein-fusion.

In a preferred embodiment of the invention the nucleic acid molecule provided for the method is a PCR-amplified nucleic acid molecule originating from a phage display vector or, alternatively, from ribosome display, plasmid-peptide display or another genetic display system.

As further steps the method may comprise a step of induction of the peptide cleavage, and purification of the peptide by an affinity column. In a preferred embodiment of the invention the induction of the peptide cleavage is carried out by temperature and pH change.

The method is usually carried out in vivo using a suitable host system In such a system the peptide-intein-fasion is expressed in e.g. Escherichia coli cells. Other microbial or eukaryotic hosts, such as yeast cells, insect cells and mammalian cells can be used as well.

On the other hand, the method can also be carried out in vitro. In such a method the translation is done without live cells and the translation machinery is obtained usually from cell lysate or an extract of cells.

The method as generally described above can be applied for a variety of purposes in peptide design, for insance for constructing a library of peptides with random hydrophilic amino acids to improve the water solubility of the peptides, for producing peptides with unnatural amino acids, or for producing a pool of peptides to be screened for improved properties.

A specific application of the method is production of any peptide obtained by phage display, in which case a pair of universal intein oligonucleotide primers are designed, whose structures enable amplification of a peptide insert without the knowledge of the peptide sequence.

We thus designed the following universal primers.
(1) Intein Fwd SapI primer having the sequence: CCT TTC TGC TCT TCC AAC GCC GAC GGG GCT. This primer will add amino acids ADGA from the phage to the peptide.
(2) Intein Rev PstI primer having the sequence: ACT TTC AAC CTG CAG TTA CCC AGC GGC CCC. This primer will add amino acids GAAG from the phage to the peptide.

These primer sequences can be used to amplify and clone any phage display peptide as an intein fusion. Briefly, the phage peptides are amplified using PCR and the inserts digested with SapI and PstI restriction enzymes. The peptide inserts are ligated to similarly digested intein vectors. The ligated vectors are transformed into host cells, and expressed. As a further step the method may comprise the step of purifying the peptides obtained from the host cells.

Our studies extend the utility of intein system to the production of small molecular weight peptides and their modification with unnatural amino acids. The possibility to incorporate unnatural amino acids such as fluorinated tryptophan should facilitate development of peptides with enhanced activity and/or stability to be used further in the drug discovery process. Furthermore, by using modified strains with multiple amino acid auxotrophies, one could replace several amino acids with unnatural ones.

In a preferred embodiment for preparing a peptide containing an unnatural amino acid, such a peptide is directly selected using phage display in an auxotrophic host and, subsequently, the selected peptide is expressed as an intein fusion on a phage.

This unnatural amino acid display system is fully compatible with the existing phage libraries made into fUSE5 vector, as the incorporation of the amino acid analogues is independent of specific codons. Thus, new libraries containing amino acid analogues can be simply generated through infection with the existing libraries. This circumvents the tedious cloning and transformation step needed for library making. Furthermore, the intein-assisted peptide expression efficiently complements the phage display system, e.g., the fluorotryptophan-containing peptides can be directly expressed as soluble. peptides for activity analysis.

This system may work best with the rare amino acids such as tryptophan. The ability to incorporate tryptophan analogues into phage libraries is important because tryptophan is very often enriched in the peptides selected by phage display.

### DETAILED DESCRIPTION OF THE INVENTION

Abbreviations:

CTT: CTTHWGFTLC peptide (Koivunen et al., 1999a)
iCTT: recombinant CTTHWGFTLC peptide;
STT: STTHWGFTLS peptide;
MMP: matrix metalloproteinase;
5OH-Trp: 5-hydroxytryptophan;
5F-Trp: 5-fluorotryptophan;
6F-Trp: 6-fluorotryptophan;
7A-Trp: 7-azatryptophan.

### DESCRIPTION OF THE FIGURES

FIG. 1A and 1B. Inhibition of NWP-2 and MMP-9 by synthetic or intein-produced peptides. (A) MMP-9 was treated with CTT, iCTT, or STT at the peptide concentrations indicated. MMP-9 activity was determined using biotinylated gelatin. (13) The activity of the alanine mutant peptides (see Table 1) was compared to that of CTT, which inhibited MMP-2 by 100% in the gelatin degradation assay. In all assays the peptides were preincubated with the enzyme for 30 min before the substrate was added. The results show means ±SD from triplicate measurements and are representative from at least two independent experiments.

FIG. 2A, 2B, 2C and 2D. Incorporation of the tryptophan analogues in the intein-CTT peptide fusion protein. (A) Tryptophan analogues used in this study (13) 12% SDS-PAGE showing the urea solubilized bacterial lysates of intein-CTT fusions. All samples were induced with IPTG. The apparent molecular weight of the intein-CTT fusion is about 30 kDa. (C) Ultraviolet absorption spectra of normal tryptophan and 50H-Trp containing CTT peptides. The absorbance spectra of the 5F-Trp and 6F-Trp containing peptides are similar to that of the wild type peptide and are not shown. (1)) Fluorescence emission spectra of the CTT peptides containing tryptophan analogues. The fluorescence emission maxima of the peptides were normalized to the same value.

FIG. 3A and 3B. Activity and stability of the tryptophan analogue containing CTT peptides. (A) Inhibition of MMP-2 using β-casein (21 IcDa) as substrate. The peptides (100 μM) were incubated with MMP-2 and β-casein (0.1 mg/ml) for 2 h and samples run on a 15% SDS-PAGE gel. (B) iCTT, 5F-CTT or a negative control peptide CERGGLETSC at a 150 μM concentration was incubated in undiluted human serum for the indicated time periods at 37° C. Samples were withdrawn, stored frozen and blotted on a nitrocellulose membrane. The level of CTT was quantitated using polyclonal anti-CTT antibody. The blots were scanned and the results shown are means ±SD from triplicate measurements. The results were similar in two other experiments.

FIG. 4A and 4B. Peptide inhibition of HT-1080 tumor cell invasion examined in 10% heat-inactivated fetal calf serum (A) or 10% non-heated normal human serum (B). The cells were allowed to migrate through the Matrigel-coated Transwells for 16 h in the absence or presence of the recombinant peptides at a 150 μM concentration. The results show means ±SD from triplicate wells. The results are representative from three independent experiments. An asterisk (*) indicates statistically significant difference (p<0.05) in Student's t-test

FIG. 5. Inhibition of gelatiaase A (MMP-2) with CTT-peptide, with pool 1 and pool 2, both containing 10 different peptide derivates of CTT. The library contains 216 different combinations of CTT peptide derivates. Clone 4 from pool 1 and clone 7 from pool 2 were purified and tested also for their inhibition activity against gelatinase A.

FIG. 6. A peptide insert from a phage clone #43 specifically binding to proMMP-9 was cloned with the universal intein oligonucleotide primers and the intein-peptide fusion was expressed and the peptide purified with HPLC. Microtiter wells were coated with 20 ng/well proMMP-9, blocked with bovine serum albumin, and the phages were allowed to bind in the presence or absence of 15 μM peptides. Unbound phages were washed with TBS-Tween and bound phage detected with anti-phage antibody-HRP conjugate. The expressed and purified peptide #43 inhibited binding of phage bearing the same peptide #43 to proMMP-9, but not the binding of another proMMP-9-binding phage bearing a different peptide (peptide #63). Similarly, peptide insert #63 inhibited only the binding of the phage bearing peptide #63 but not the phage bearing peptide #43. The peptide CTT had no effect on the binding of phages #43 and #63, confirming the specificity of the phage binding.

FIG. 7. Schematic representation of the strategy to prepare the auxotrophic phage host strains.

FIG. 8. Structures of the amino acid analogues tested for phage incorporation. FIG. 9A and 9B. Phage production in the presence of the amino acid analogues. The phages were prepared as described in the methods. Serial dilutions of the culture supernatants were made in triplicate and these were used to infect E. coli K91/kan. The percentage of infective phage compared to phage culture in the presence of the parental amino acid is shown. Representative data from the phage production in the presence of tryptophan (A) and methionine (B) analogues are shown.

FIG. 10. Fluorotryptophan incorporation changes the intrinsic fluorescence properties of phages. The phage samples were denaturated by heating in 1% SDS containing buffer and the fluorescence spectra recorded were recorded with an excitation at 295 nm. Fluorescence emission was measured in the range of 300-500 nm.

FIG. 11. Enrichment of fluorophage library after two rounds of biopanning with human cell lines Eahy926 and KS 1767. The phages were subtracted with Eahy926 cells and selected for KS1767 binding.

Experimental

Methods

Chemical peptide synthesis. Peptides were synthesized with an Applied Biosystems model 433A (Foster City, Calif.) using Fmoc-chemistry as reported previously (Koivunen et al., 1999a), except that disulfide bond formation was done using hydrogen peroxide. Briefly, the peptide was dissolved in 50 mM ammonium acetate (pH 7.5) at a 1 mg/ml concentration and 0.5 ml of 3% hydrogen peroxide per 100 mg peptide was added. After 30 min incubation, pH was adjusted to 3.0 and the cyclized peptide was purified by reverse-phase BPLC using a linear acetonitrile gradient (0%→70% during 30 min) in 0.1% trifluoroacetic acid.

Cloning of the intein-peptide fusions. A synthetic oligonucleotide 5′-GGTGGTGCTCTTCCAACTGTACGACCCATTGGGGATTTACTTTATGTTAACTGCAGGCG-3′ encoding the CTTHWGFTLC peptide was converted to double stranded form using Dynazyme II DNA polymerase (Finnzymes, Espoo, Finland) with a primer 5′-CGCCTGCAGTTAACA-3′, and digested with SapI and PstI. Purified insert was ligated in frame to SapI-PstI-digested pTwin vector backbone (New England Biolabs) (Evans et al., 1999). The presence of the correct insert was verified by sequence analysis. Similar cloning strategy was used to prepare the alanine-mutant peptides using codon GCG for alanine. For the cloning of any phage peptide inserts, universal oligonucleotides 5′-CCT TTC TGC TCT TCC AAC GCC GAC GGG GCT-3′(Intein Fwd SapI), 5′-ACT TTC AAC CTG CAG TTA CCC AGC GGC CCC-3′ (Intein Rev PstI) were used. For the hydrophilic CTT peptide library, a synthetic degenerate oligonucleotide 5′-GGTGGTTGCTCTTCCAACGGCCGCCVAVVAVTATVAVGGCTGTACCACCCATTTACTTTATGTTAACTGCAGGCG-3′ (where V is A, C or G) was prepared, and converted to double-stranded DNA with the same primer as the normal CTT peptide.

Peptide production in bacteria. The plasmids encoding intein fusion peptides were transformed into E. coli ER2566 strain (New England Biolabs). The clones were cultured in LB medium containing 100 μg/ml ampicillin until OD₆₀₀ was 0.7. The protein expression was induced with 0.3 mM IPTG and incubation continued for 4 h at 37° C. The bacterial pellets were suspended in 20 mM Tris-HCl (pH 8.5)/500 mM NaCl/1 mM EDTA/1% Triton X-100 (Buffer Bi). Following sonication and centrifugation, the soluble fraction was applied on a chitin affinity column (New England Biolabs). The insoluble fraction containing most of the intein-fusion protein was solubilized with 8 M urea/100 mM Tris-HCl (pH 8.0)/100 mM NaCl/2 mM EDTA and sonicated. The solubilized material was subsequently diluted at least 1:16 with the buffer B1 without Triton X-100 and cleared by centrifugation. The clarified supernatant was also applied on the chitin column. The column was washed extensively with buffer B1 lacking Triton X-100. The intein-cleavage reaction was performed on-column by overnight incubation in 50 mM ammonium acetate/1 mM EDTA (pH 7.0) at 22° C. The free peptide was eluted, concentrated by lyophilization or by Sep-Pak C18 cartridges (Waters) and purified with reverse-phase HPLC. The identity of each peptide was verified by MALDI-TOF mass spectrometry. Peptides were quantified using o-phthalaldehyde or HPLC analysis. Known concentrations of the CTT peptide were used as standards.

Generation of tryptophan auxotrophic E. coli ER2566 mutant. In vitro assembled bacteriophage Mu DNA transposition complexes were prepared essentially as described previously (Lamberg et al., 2002). Briefly, 1.1 pmol transposon DNA containing a kanamycin resistance gene and 4.9 pmol MuA protein were mixed in 20 μl of 150 mM Tris-HCl (pH 6.0)/50% glycerol/0.025% Triton X-100/150 mM NaCl/0.1 mM EDTA. The transposition complex assembly reaction was carried out at 30° C. for 2 h. The complexes were electroporated as 1:8 or 1:16 dilutions into electrocompetent E. coli ER2566 and plated on LB plates containing 50 μg/ml kanamycin. The clones obtained were replica-plated on M9 minimal plates and M9 plates containing 1 mM DL-tptophan (Sigma). A clone named ER2566/Trp82 requiring Trp for growth was chosen for further studies. To determine the transposon insertion site, the chromosomal DNA was isolated with genomic DNA isolation kit (Qiagen) and digested with PstI. The resulting genomic fragments were ligated with PstI digested pUC19 plasmid and transformants selected in the presence of kanamycin. The DNA sequences of transposon borders were determined by sequencing with transposon specific primers 5′-ATCAGCGGCCGCGATCC-3′ and 5′-TTATTCGGTCGAAAAGGATCC-3′. The genomic location of the insertion was identified using the BLAST search.

Generation of auxotrophic E. coli for amino acid analogue incorporation into phage particles. In vitro assembled bacteriophage Mu DNA trasposition complexes containing a kanamycin resistance gene were prepared and electroporated into MC1061 as described previously (Lamberg et al., 2002). Successful transpositions were identified by gain of kanamycin resistance and the resulting colonies were screened for auxotrophism by replica-plating on M9 minimal agar plates containing 0.5 mM L-leucine, 1 mM thiamine in the absence or presence of 0.5 mM methionine or tryptophan. Clones requiring Met or Trp for growth were selected for the incorporation studies. To allow phage infection, F′-pilus [lacIq L8 pro with Tn9 in lacYZ] from the E. coli strain NK5468 (E. coli Genetic Center, Yale University, New Haven, Conn.) was transferred by mating. Successful matings were identified by the acquisition of a chloramphenicol resistance.

Incorporation of tryptophan analogues into peptides. The plasmid coding for intein-CTT fusion was transformed into the auxotrophic ER2566/Trp82. The clone was cultured in M9 medium supplemented with 0.6% glycerol, 0.1 mM CaCl₂, 2 mM MgCl₂, 0.01 mM FeSO₄, 100 μg/ml ampicillin, 25 μg/ml kanamycin and 0.5 mM DL-tryptophan until OD₆₀₀ reached 0.8-1.0. Incorporation of the tryptophan analogues 5-hydroxy-L-tryptophan (5OH-Trp, Sigma), 5-fluoro-DL-tryptophan (SF-Trp), 6-fluoro-DL-tryptophan (6F-Trp) and DL-7-azatryptophan (7A-Trp, ICN Biomedicals) was accomplished by a medium shift procedure (Minks et al., 1999; Mohammadi et al., 2001; Ross et al., 1997; Tang et al., 2001). The bacteria were centrifuged and suspended in fresh M9 medium lacking tryptophan or analogues. The bacteria were grown for 15 min at 37° C. to exhaust most of remaining tryptophan, and the typtophan analogue was then added at a 0.5. mM final concentration together with 0.5 mM IPTG. After 4 h cultivation at 37° C., the bacteria were pelleted, and the fusion protein purification was done as above.

Gelatinase inhibition assays. Gelatiases proMMP-2 and proMMP-9 (Roche) were activated with p-aminophenylmercuric acetate or trypsin, respectively, and then incubated in the presence or absence of each peptide to be tested for 30 min. The gelatinase inhibitory activity was determined using the following three assays: (i) The degradation of biotinylated gelatin was examined using a gelatinase activity kit according to the manufacturer's instructions. (Roche). (ii) The degradation of a MMP-2 specific fluorescent peptide substrate MCA-Pro-Leu-Ala-Nva-Dpa-Ala-Arg-NH₂ (Calbiochem) (2.5 μM final concentration) was followed using MOS-250 spectrofluorometer (Bio-Logic SA, Claix, France) with 330 nm excitation and 390 nm emission. (iii) The degradation of β-casein was studied by incubating activated MMP-2 with 0.1 mg/ml concentration of β-casein for 2 h at 37° C., after which the samples were analyzed on a 15% SDS-PAGE gel.

Cell invasion. ET-1080 human fibrosarcoma cells were cultured in DMEM medium containing 10% fetal calf serum and supplemented with penicillin, streptomycin and L-glutamine. Cell invasion assay was conducted using Matrigel coated invasion chambers in the serum-containing medium as described (Koivunen et al., 1999a). Briefly, the cells were preincubated with the peptides for I h and then allowed to migrate through the Matrigel coated invasion chambers (Becton Dickinson) for 16 h. The migrated cells were stained with crystal violet and counted.

Spectrometry and fluorometry. The absorbance spectrum for each peptide in the 200-375 nm range was measured in 20 mM Tris-HCl (pH 7.4)/50 mM NaCl/0.1 mM EDTA with Genesys 5 spectrophotometer (Thermo Spectronic, Rochester, N.Y.). The fluorescence spectra of the CTT phage cultured in the presence of tryptophan, 5FW or 6FW were measured from heat-denatured phages (2×10⁹/ml) in 10 mM Tris-HCl (pH 7.5)/140 mM NaCl/1% SDS. The fluorescence emission spectra (average of three scans) at 300-500 nm were recorded with MOS-250 spectrofluorometer. The peptides were excited at 295 nm (bandwidth 5 nm) and the emission spectra recorded in the 300-500 nm range. Peptide stability in human serum. Blood samples were collected from the laboratory personnel and the sera stored in aliquots at −70° C. The peptides were added to the undiluted human serum at the final concentration of 150 μM. The serum was incubated at 37° C. and aliquots were taken at different time points, diluted in PBS/0.05% Tween 20, and immediately frozen in liquid nitrogen. The samples were thawn and loaded on a nitrocellulose membrane using a 96-well dot blotter. Following blocking with 5% BSA in TBS/0.05% Tween 20, the membrane was incubated with a 1:500 dilution of anti-CTT rabbit serum which was prepared by immunizing with the CTT-peptide coupled to keyhole limpet hemocyanin (Sigma). Bound anti-CTT antibody was detected by enhanced chemiluminescence using peroxidase-conjugated anti-rabbit antibody (DAKO, Denmark), at a 1:2000 dilution.

Amino acid analogues. DL-ethionine (Eth), DL-norleucine (Nle), 4-aza-DL-leucine (Ale), 5-hydroxy-L-tryptophan (50H), 5-fluoro-DLtryptophan (5FW), 6-fluoro-DL-tryptophan (6FW), and DL-7-azatryptophan (7AW) were from Sigma-Aldrich or ICN Biomedicals. Incorporation of amino acid analogues into phage particles. The filamentous bacteriophage fuSE5 displaying the CTT peptide was cultured in MB5F or MB64F strains in a chemically defined M9 medium supplemented with 0.2% glucose, 0.1 mM CaCl₂, 2 mM MgCl_{2,} 0.01 mM FeSO₄, 20 μg/ml tetracycline, 25 μg/ml kanamycin, 10 μg/ml chloramphenicol, 1 mM thiamine, 0.2 mM each of guanosine, uracil, adenine and thymidine, and all the twenty amino acids in a 0.1-0.8 mM concentration (Neidhardt et al., 1974). Incorporation of the amino acid analogues was accomplished by a medium shift procedure. Briefly, the bacteria (OD600=0.7-1.0) were centrifuged and suspended in a fresh M9 medium lacking the amino acid to be replaced. The analogues were added at a 0.5-2 mM final concentration (as the L-isomer) and the bacteria were cultured overnight

Phage quantitation. Serial dilutions of the phage supernatants were prepared and these were used to infect the E. coli K91/kan strain using standard techniques (Koivunen et al., 1999b). Ten μl aliquots of the infections were plated in triplicates on LB agar plates containing 40 μg/ml tetracycline and 10 μg/ml kanaycin. After an overnight incubation, the number of bacterial colonies was counted.

Preparation of a fluorophage library. 15 μL aliquots of CX₇C, CX₈C and X₉C libraries (Koivunen et al., 1999a, Koivunen et al., 2001) were infected to MB5F strain cultured in Terrific Broth. The phage infection resulted in 0.5×10⁹ individual clones. After an overnight incubation, the bacteria were subcultured into one litre M9 medium and cultured overnight. The medium shift procedure was performed as above and the 5FW and 6FW analogues were added simultaneously to 0.5 mM final concentration. The next day resulting phage were precipitated twice with polyethylene glycol PEG)/NaCl (Koivunen et al., 1999b).

Biopanning with the fluorophage library. A total of 2.5×10⁵ Eahy926 cells (Koivunen et al., 1999a, Koivunen et al., 2001) were suspended in 150 μl 1% bovine serum albumin in DMEM medium and incubated with an aliquot of the fluorophage library (1×10⁹ transducing units) for 4 h at +4° C. The cells were centrifuged through a bovine serum gradient (Williams et al., 2002) and the resulting phage supernatant was applied to 2.5×10⁵ KS1767 cells and incubated for 4 h at +4PC. The sample was centrifuged again through a serum gradient and the cell pellet was used to infect MB5F bacteria. The bacteria were grown overnight and then for another day in the presence of fluorotryptophans. The phage were collected and used for a second round of subtraction with Eahy926 cells and selection with KS1767 cells.

Results

Peptide Biosynthesis Using an Intein Vector

We chose the Ssp DnaB nini-intein with C-terminal cleavage activity for the peptide production, as this intein contains only 154 amino acids and the C-terminal protein fusions are typically efficiently expressed. Furthermore, the cleavage activity of this intein is induced by a pH and temperature change from pH 8.5 and 4° C. to pH 7.0 and 22° C. We could thus avoid the thiol-induced intein cleavage, which might interfere with the disulfide bonding and the activity of a peptide. One of the advantages for using an intein for the production of peptides with cysteines in the termini is that cysteine is a catalytically favorable amino acid resulting in high cleavage efficiency (Paulus, 2000).

We began our studies by examining intein-mediated production of the ten-residue long gelatinase inhibitor peptide CTT, which is active only in the cyclic disulfide form. Essentially all intein-CTT fusion protein was found in inclusion bodies and was recovered by solubilization in urea. The peptide could be obtained in 70-90% purity after the on-column cleavage reaction. The yield after the final HPLC purification was up to 2 mg peptide from one liter of bacterial culture. The intein-fusion derived CTT (iCTT) was spontaneously cyclized having the expected molecular weight of the disulfide bond-containing CTT peptide in mass spectrometry (Table 1). Next, alanine-scanning mutagenesis of CTT was carried out to identify amino acid residues required for the gelatinase inhibitory activity. The Ala-substituted peptides were obtained with similar yields as iCTT. Mass spectrometry confirmed the identity of each cyclic peptide (Table 1).

TABLE 1

Recombinant peptides prepared
by the intein system.

Mass, Da

| | | | |
|---|---|---|---|
| Peptide | Sequence | Calc.* | Obs. |

| | | | |
|---|---|---|---|
| iCTT | CTTHWGFTLC | 1166.4 | 1166.5 |
| T1 → A | CATHWGFTLC | 1136.3 | 1136.4 |
| T2 → A | CTAHWGFTLC | 1136.3 | 1136.4 |
| H → A | CTTAWGFTLC | 1100.3 | 1100.5 |
| W → A | CTTHAGFTLC | 1051.2 | 1051.4 |
| G → A | CTTHWAFTLC | 1180.4 | 1180.5 |
| F → A | CTTHWGATLC | 1090.3 | 1090.4 |
| T3 → A | CTTHWGFALC | 1136.3 | 1136.5 |
| L → A | CTTHWGFTAC | 1124.3 | 1124.4 |
| 5OH-CTT | CTTH(5OHW)GFTLC | 1182.4 | 1182.4 |
| 5F-CTT | CTTH(5FW)GFTLC | 1184.4 | 1184.3 |
| 6F-CTT | CTTH(6FW)GFTLC | 1184.4 | 1184.2 |

*molecular weight calculated for the oxidized, cyclic form

Functional Analysis of the Gelatinase Inhibitory Peptides

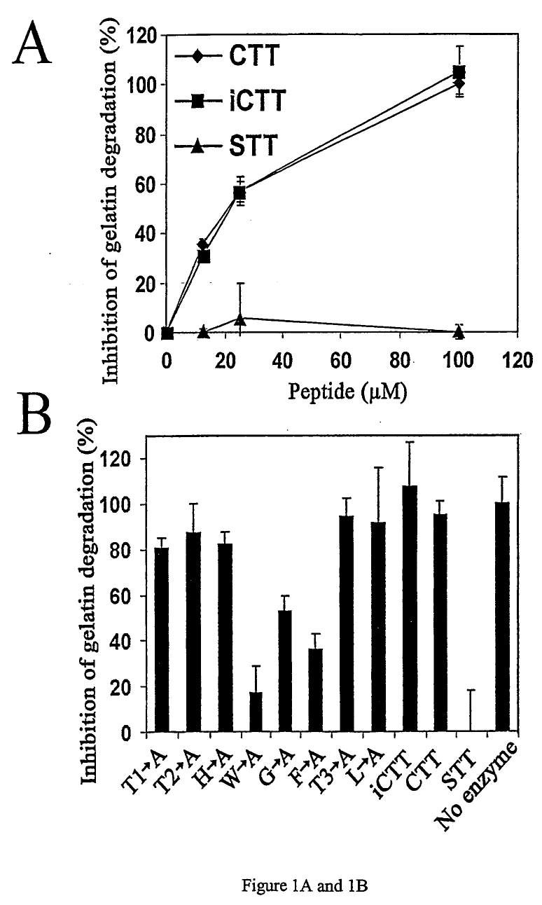

The gelatinase inhibitory activity of iCTT was found to be identical with that of the chemically synthesized CTT in several assays. In gelatin degradation assay, iCTT and CTT exhibited a similar dose dependency, the IC₅₀ values being 20 μM for both MMP-2 and MMP-9 inhibition (FIG. 1A and data not shown). The non-cyclic synthetic control peptide STTHWGFTLS (STT) was several fold less active than iCTT.

Analysis of the Ala-substituted peptides in the gelatin degradation assay showed that changes of tryptophan, glycine and phenylalanine significantly decreased the gelatinase inhibitory activity (FIG. 1B). The W→A, G→A and F→A mutant peptides had 17±12, 53±7 and 36±7% of the activity in comparison to the wild type peptide, respectively. Ala-replacements at all the other positions did not appreciably affect the gelatinase inhibitory activity. For example, the peptide with H→A substitution retained about 80% of the wild-type activity. Similar results were obtained by comparison of the peptides in the fluorogenic MMP-2 substrate assay (data not shown).

Biosynthesis of Peptides Containing Tryptophan Analogues

Having demonstrated the capability of the intein expression for peptide synthesis, we examined the possibility of incorporating unnatural amino acids into intein-derived peptides. We focused our studies on the modification of the single tryptophan residue of CTT as it seemed to offer the best possibilities to modulate the activity and possibly the stability of the peptide. A tryptophan auxotrophic E. coli required for efficient incorporation of unnatural tryptophan analogues was prepared by mutagenesis using in vitro assembled bacteriophage Mu DNA transposition complexes. A clone designated as ER2566/Trp82 was isolated and found to be auxotrophic for tryptophan. The transposon insertion site was in the genomic location duplicating the nucleotides 1315340-44, numbered according to completely sequenced E. coli K12 strain. Thus, the correct five base pair target site duplication characteristic to Mu transposition was identified (Lamberg et al., 2002). The insertion was within the trpB gene encoding for tryptophan synthase β subunit, consistent with the observed phenotype.

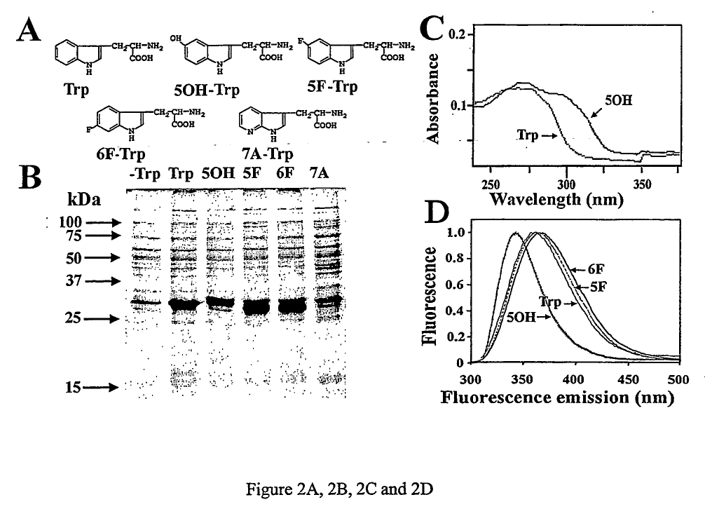

The Er2566/Trp82 clone was used to express CTT intein fusion with 5-hydroxytryptophan, 5-fluorotryptophan, 6-fluorotryptophan or 7-azatryptophan added into the bacterial culture (FIG. 2A). Previously, these tryptophan analogues have been incorporated into several proteins synthesized by tryptophan auxotrophic E. coli is et al., 1999; Mohammadi et al., 2001; Ross et al., 1997). There are seven tryptophan residues in the expressed intein fusion protein, three in the chitin-binding domain, three in the intein and one in the CTT peptide. We observed incorporation of all four tryptophan analogues into the intein fusion protein (FIG. 2B). On SDS-PAGE, the intein-fusion protein containing fluorinated Trp analogues migrated faster than the protein with normal tryptophan. The more polar 5OH-Trp and 7A-Trp residues on the other hand caused the fusion protein to migrate slower. The yields of the fusion proteins were slightly reduced (5OH-Trp, SF-Trp and 6F-Tip) or significantly reduced (7A-Trp) as compared with the wild type protein. Notably, 5F-Trp and 6F-Trp did not significantly impair the intein cleavage activity and the CTT peptides containing fluorinated Trp were obtained at about 0.3 mg yield per liter minimal medium. The 50H-Trp residue affected the intein cleavage causing reduced peptide yields. 7A-Trp containing peptide was not obtained in amounts sufficient for activity determination. Mass spectrometry confirmed that each peptide contained the expected unnatural tryptophan analogue (Table 1). Only a minor amount of wild type CTT peptide was present in fluorinated peptide preparations and no wild type peptide at all could be detected in the 5OH-CTT preparation (data not shown). The identity of the modified peptides could be further confirmed by ultraviolet absorption and fluorescence spectroscopy. The 5OH-Trp containing CTT peptide had a characteristic absorbance profile with a distinct red-shifted secondary absorption maximum (FIG. 2C). All modified peptides differed in their fluorescence emission spectra from the wild type peptide (FIG. 2D).

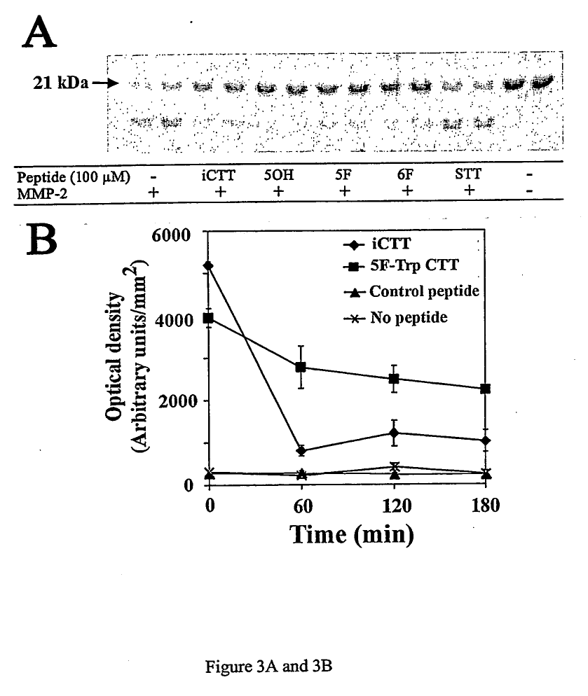

The gelatinase inhibitory activities of the modified CTT peptides were tested in the β-casein degradation assay (FIG. 3A) and the gelatin degradation assay (data not shown). No significant differences in the gelatinase inhibitory activity were seen in these assays. At a 100 μM concentration 5OH-Trp, 5F-Trp and 6F-Trp containing peptides inhibited MMP-2 with a similar efficiency as iCTT does with nearly complete inhibition of casein degradation. As amino acid analogues can contribute to the protease sensitivity of the peptides, we next studied the stability of the peptides by incubating them in normal human serum. To determine the peptide levels, we used anti-CTT antibody that also recognized the 5F-Trp containing peptide. The 5F-Trp containing peptide was more stable in serum with a half-life of 3 hours in comparison to the 0.5 hour half-life of the wild type CTT peptide (FIG. 3B). We could not determine the half-lives of 5OH-Trp and 6F-Trp containing peptides as the anti-CTT antibody recognized these peptides only weakly in the presence of serum. The CTT antibody was highly specific as cyclic control peptides CERGGLETSC and CPCFLLGCC did not react with the anti-CTT antibody. No difference between the stability of CTT and 5F-Tip containing peptide was seen in cell culture medium supplemented with 10% heat-inactivated fetal calf serum (data not shown).

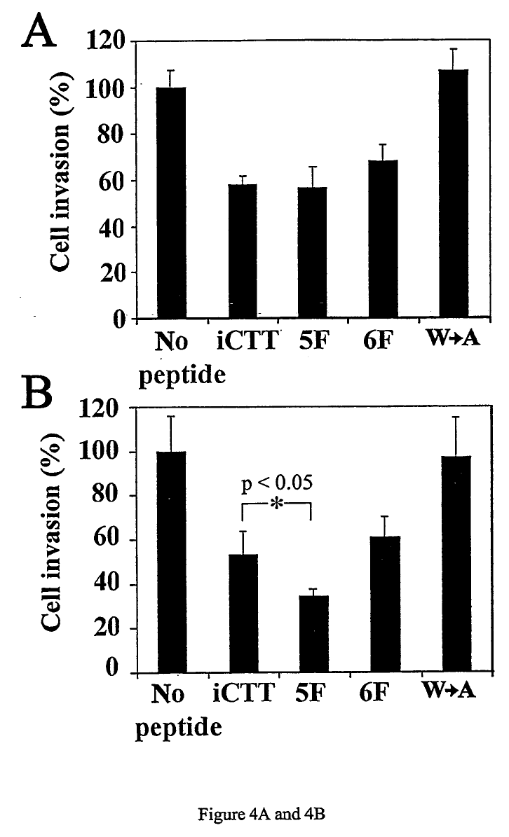

In the cell invasion assay, the 5F-Trp and 6F-Trp containing peptides displayed an activity similar to that of iCIT when the cells were cultured in the presence of 10% heat-inactivated fetal calf serum (FIG. 4A). However, when the HT-1080 cells were cultured in 10% human serum that had not been heat-inactivated, the 5F-Trp containing peptide was significantly better inhibitor of cell invasion than iCTT (FIG. 4B), correlating with the serum stability data. This effect was specific for fluorine substitution at 5-position of Trp as the activity of the 6F-Trp containing peptide did not differ from that of the wild type peptide.

Preparing a Hydrophilic Intein Peptide Library

To achieve labeling of the CTT peptide with radioactive iodine, peptides with additional tyrosine were prepared by chemical synthesis. However, these peptides were found insoluble in water, although the CTT peptide itself is water-soluble. Thus, we prepared a peptide library with intein system to screen for a water-soluble tyrosine-containing CTT peptide. A degenerate oligonucleotide having randomized amino acids coding for polar amino acids were used to potentially enhance the solubility of the tyrosine containing CTT peptide. The resulting library coded for peptides GRXXYXGCTIHWGFTLC, wherein X is any hydrophilic amino acid. First an oligonucleotide 5′-GGTGGTTGCTCTTCCAACGGCCGCCVAVVAVTATVAVGGCTGTACCACCCATTACTATATGTrAACTGCAGGCG-3′ was designed, and prepared by combinatorial synthesis using an oligonucleotide synthesizer. The oligonucleotide contained three VAV codons, (wherein V is G or A or C), which code for hydrophilic amino acids.

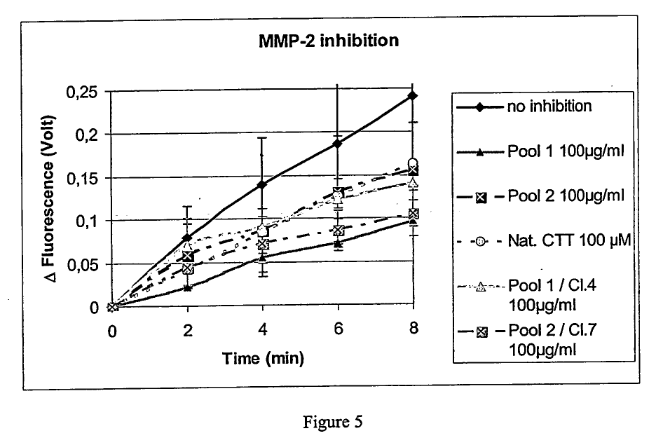

The oligonucleotide was made double-stranded using PCR. The PCR product was digested with PstI and SapI and cloned to TWIN2 intein vector (New England Biolabs), digested also with PstI and SapI. This DNA construct was electroporated into MC1061 competent cells. The library obtained contained 216 variations of the CTT-peptide. The plasmid vectors were extracted from the pool of at least 216 independent clones of MC1061. The plasmids were then electroporated to ER2566 cells enabling the production of inteins. The cells harboring the plasmids were plated on LB plates containing ampicillin. 10 independent clones were pooled to one single pool. These 10 clones in one pool were cultured and the peptides were expressed and purified with chitin affinity column and reverse-phase C18 column. Each pool of peptides was tested for activity. The solubility in PBS was also tested. Two clones of two pools with distinct activity and good solubility were cultured, and the peptides were purified as described above. One of these peptides (having the sequence GRENYHGCTTRWGFTLC) was more soluble into water or PBS than the original peptide, and it was active (FIG. 5). The plasmid coding for this peptide was sequenced and the peptide was synthesized with chemical synthesis.

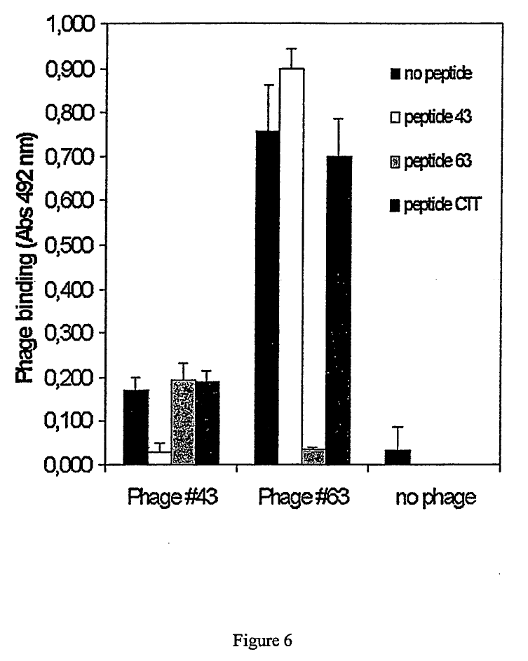

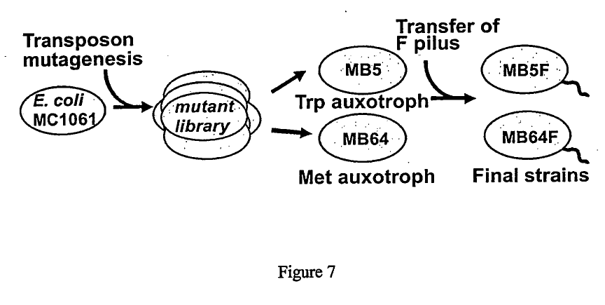

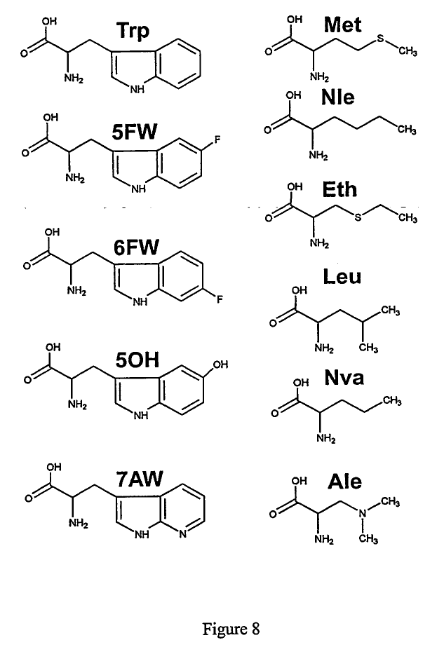

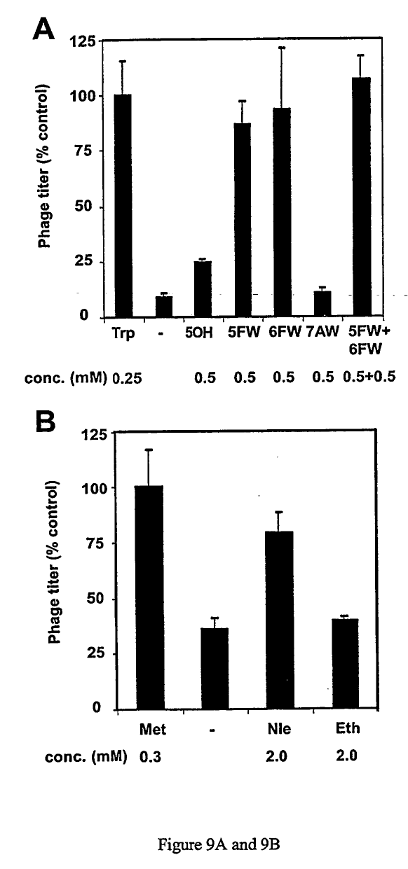

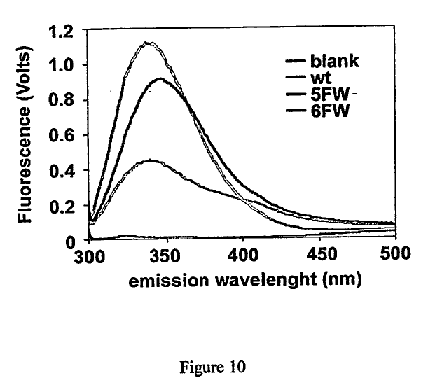

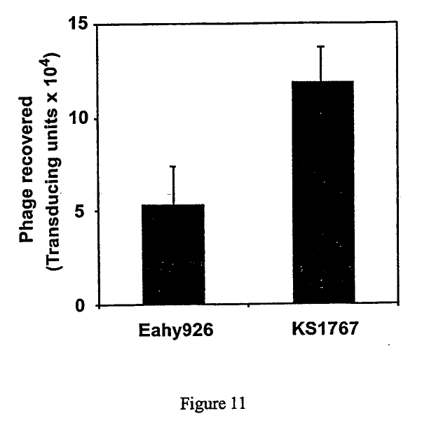

To demonstrate the utility of the general strategy to express any phage display peptide as an intein fusion, universal primers were used to amplify peptides from phages selected for binding to proMMP-9. The peptide inserts from phage clones specifically binding to proMMP-9 after three rounds of phage selection were amplified and cloned into an intein vector. The peptides were expressed, and purified with HPLC. The specificity of the phage binding to proMMP-9 and the identity of the peptides was confirmed by phage ELISA. The peptides expresses by the intein system only blocked the binding of phage with the same peptide insert, but did not compete with the binding of phage bearing another insert (FIG. 6).

Incorporating Amino Acid Analogues into Phage Particles

To achieve a high-level phage production together with efficient incorporation of the amino acid analogues, we mutagenized the E. coli strain MC1061 commonly used for the preparation of phage display libraries. Auxotrophic derivatives for tryptophan and methionine were isolated by random insertional mutagenesis using in vitro assembled bacteriophage Mu DNA transposition complexes according to Lambert et al. 2002. The parental strain MC1061 does not have the F pilus required for the phage infectivity, thus we transferred the F′ pilus from the E. coli strain NK5468 to the newly isolated auxotrophic strains. This resulted in the tryptophan auxotroph strain designated MB5F and the methionine auxotroph MB64F. FIG. (7) illustrates the strategy to obtain the bacterial strains for the peptide display system. The new strains were functional as they could be infected with the filamentous phage, although the infectivity was about 10% compared to the K91/kan host.

The commercially available tryptophan analogues 5-hydroxy-L-tryptophan (50H), 5-fluoro-DL-tryptophan (5FW), 6-fluoro-DL-tryptophan (6FW) and DL-7-azatryptophan (7AW) were first tested for the incorporation efficiency into phage particles. FIG. (8). The tryptophan auxotrophic MB5F strain was infected with fUSE5 phage carrying the CTTHWGFTLC peptide. The infected bacteria were first cultured in a defined medium and then shifted to a medium containing the amino acid analogue to be tested. As a control, the bacteria were transferred to a medium lacking tryptophan or the analogues. After an overnight culture, the titer of the CTT-fUSE5 phage in the culture supernatant was measured by infecting the E. coli K91/kan. Roughly equal number of infective particles was obtained with 5FW and 6FW as compared to tryptophan. The combination of 5FW and 6FW also yielded phage particles efficiently. In contrast, the 50H and 7AW supported the phage production poorly. FIG. (9A).

Methionine analogues ethionine and norleucine were similarly tested for the incorporation efficiency using the strain MB64F. In these experiments norleucine (Nle), but not ethionine (Eth) could be incorporated with a good efficiency. FIG. (9B). Leucine analogues norvaline (Nva) and 4-azaleucine (Ale) were also tested, as the MC1061 strain is naturally auxotrophic for leucine. However, attempts to incorporate these analogues were unsuccessful (data not shown).

The analogue incorporation efficiency could not be studied using the differential migration of tryptophan analogues in SDS-PAGE due to insufficient resolution of the major coat protein pVm, which has only 50 amino acids and a molecular weight of about 5200. To demonstrate that the fluorinated tryptophan analogues were indeed incorporated into phage particles, we took advantage of the fact that the fluorine substitutions change the intrinsic fluorescence properties of tryptophan in proteins. Phages cultured in the presence of 5FW and 6FW were precipitated with PEG/NaCl for four times to remove any unincorporated fluorotryptophan. The resulting phages (2×10⁹ phages/ml) were denatured with SDS and the fluorescence spectra recorded. The observed fluorescence emission is mainly derived from the single tryptophan in the major coat protein pVHII, which is present in about 2800 copies/virion whereas the other coat proteins are present in 2-5 copies. When the samples are excited with 295 nm wavelength, the fluorescence quantum yield of SFW and 6FW-containing phages is highly enhanced in accordance with previous data with these analogues (Minks et al., 1999). Furthermore, the emission maximum of 6FW containing phage preparation is shifted 6 nm to 345 nm, compared to the 339 nm emission maximum of the wild type phage. FIG. (10). These results indicate significant incorporation of the analogues, although quantitative analysis of the incorporation level is not possible.

To demonstrate that unnatural amino acid-containing phage libraries can be prepared and used in biopanning, we prepared CX₇C, CX₈C and X₉C libraries containing SFW and 6FW. The libraries were obtained by infecting the MB5F strain with the wild-type CX₇C, CX₈C and X₉C libraries (Koivunen et al., 1999a; Koivunen et al., 2001) followed by culturing of the phage-infected bacteria in the presence of 5FW and 6FW. The amplified phage were biopanned with the KS1767 human Kaposi's sarcoma cells to isolate peptides recognizing these tumor cells, but not the endothelial cell line Eahy926. KS1767-specific peptides were obtained after subtracting the library of the peptides binding to Eahy926 cells FIG. (11). After two rounds of biopanning the enrichment was 2.2-fold, which is significant in considering the relatively slow formation of phage particles in the chemically defined medium.

Sequence Listing Free Text

For SEQ. ID. No. 1-4:
Description of Artificial Sequence: Oligonucleotide primer
For SEQ. ID. No. 5:
Description of Artificial Sequence: Oligonucleotide primer
v at positions 26, 28, 29, 31, 35 and 37 is a c or g
For SEQ. ID. No. 6-7:
Description of Artificial Sequence: Oligonucleotide primer
For SEQ. ID. No. 8-11:
Description of Unknown Organism: Unknown
For SEQ. ID. No. 12-19:
Description of Artificial Sequence: Ala-substitution of the CTT-peptide
For SEQ. ID. No. 20:
Description of Artificial Sequence: CTT-peptide with a tryptophan analogue at position 5 Xaa at position 5 is 5-OH-Trp, 5-F-Trp or 6-F-Trp
For SEQ. ID. No. 21-22:
Description of Artificial Sequence: Control sequence
For SEQ. ID. No. 23:
Description of Artificial Sequence: CTT-peptide with additional hydrophilic amino acids Xaa at positions 3, 4 and 6 is any hydrophilic amino acid
For SEQ. ID. No. 24:
Description of Artificial Sequence: CIT-peptide with additional hydrophilic amino acids

### REFERENCES

Chong, S., Mersha, F. B., Comb, D. G., Scott, M. E., Landry, D., Vence, L. M., Perler, F. B., Benner, I., Kucera, R. B., Hirvonen, C. A., Pelletier, J. J., Paulus, H. and Xu, M. Q. (1997) Single-column purification of free recombinant proteins using a self-cleavable affinity tag derived from a protein splicing element. Gene, 192: 271-81.

Dwyer, M. A.; Lu, W.; Dwyer, J. J. and Kossiakoff, A. A. (2000) Chem. Biol. 7, 263-274.

Evans, T. C., Jr., Benner, J. and Xu, M. Q. (1999) The cyclization and polymerization of bacterially expressed proteins using modified self-splicing inteins. J. Biol. Chem., 274: 18359-63.

Koivunen, E., Arap, W., Valtanen, H., Rainisalo, A., Medina, O. P., Heikkila, P., Kantor, C., Gahmberg, C. G., Salo, T., Konttinen, Y. T., Sorsa, T., Ruoslahti, E. and Pasqualini, R. (1999a) Tumor targeting with a selective gelatinase inhibitor. Nat. Biotechnol., 17: 768-74.

Koivunen, E.; Restel, B. H.; Rajotte, D.; Lahdenranta, J.; Hagedorn, M.; Arap, W.; Pasqualini, R. (1999b), Methods Mol. Biol. 129, 3-17.

Koivunen, E.; Ranta, T. M.; Annila, A.; Taube, S.; Uppala, A.; Jolinen, M.; van Willigen, G.; Ihanus, E.; Gahmberg, C. G. J. (2001), Cell Biol. 153, 905-916.

Lamberg, A., Nieminen, S., Qiao, M. and Savilahti, H. (2002) Efficient insertional mutagenesis strategy for bacterial genomes involving electroporation of in vitro-assembled DNA transposition complexes of bacteriophage Mu. Appl. Environ. Microbiol., 68: 705-712.

Mathys, S., Evans, T. C., Chute, I. C., Wu, H., Chong, S., Benner, J., Liu, X. Q. and Xu, M. Q. (1999) Characterization of a self-splicing mini-intein and its conversion into autocatalytic N- and C-terminal cleavage elements: facile production of protein building blocks for protein ligation. Gene, 231:1-13.

Minks, C., Huber, R., Moroder, L. and Budisa, N. (1999) Atomic mutations at the single tryptophan residue of human recombinant annexin V: effects on structure, stability, and activity. Biochemistry, 38: 10649-59.

Mohammadi, F., Prentice, G. A. and Merrill, A. R. (2001) Protein-protein interaction using tryptophan analogues: novel spectroscopic probes for toxin-elongation factor-2 interactions. Biochemistry, 40: 10273-83.

Neidhardt, F. C.; Bloch, P. L.; Smith, D. F. (1974) J. Bacteriol 119, 736-747.

Paulus, H. (2000) Protein splicing and related forms of protein autoprocessing. Annu. Rev. Biochem., 69: 447-96.

Ross, J. B., Szabo, A. G. and Hogue, C. W. (1997) Enhancement of protein spectra with tryptophan analogs: fluorescence spectroscopy of protein-protein and protein-nucleic acid interactions. Methods Enzymol., 278: 151-90.

Tang, Y., Ghirlanda, G., Petka, W. A., Nakajima, T., DeGrado, W. F. and Tirrell, D. A. (2001) Fluorinated Coiled-Coil Proteins Prepared In Vivo Display Enhanced Thermal and Chemical Stability. Angew. Chem. Int. Ed. Engl., 40: 1494-1496.

Williams, B. R.; Sharon, J. (2002), Immunol. Lett. 81, 141-148.

1. A method for producing a peptide having at least one disulfide bridge, comprising the steps of
providing a nucleic acid molecule encoding a polypeptide comprising a peptide of interest,
incorporating said nucleic acid molecule into an expression vector as a fusion with an intein,
expressing the peptide-intein-fusion, and
inducing the peptide cleavage by temperature and pH change.

2. The method according to claim 1, further comprising the step of
purifying the peptide by an affinity column.

3. The method according to claim 1, wherein the method is carried out in vivo in a host system.

4. The method according to claim 3, wherein the host system comprises Escherichia coli cells.

5. The method according to claim 1, wherein the method is carried out in vitro.

6. The method according to claim 1, wherein the nucleic acid molecule provided is a synthetic nucleic acid molecule, comprising a nucleotide sequence encoding a peptide of interest, and elements enabling the incorporation of the nucleic acid molecule into an expression vector.

7. The method according to claim 1, wherein the nucleic acid molecule provided is a PCR-amplified nucleic acid molecule originating from a phage display vector.

8. The method according to claim 7, wherein the peptide encoded by the phage display vector contains an amino acid analogue.

9. The method according to claim 7 for preparing any peptide screened by phage display, wherein the nucleic acid molecule provided is a PCR amplicon obtained by using a pair of oligonucleotide primers flanking the nucleotide sequence encoding the peptide of interest, and containing elements required for incorporation of said sequence into an expression vector.

10. The method according to claim 9, wherein the pair of oligonucleotide primers consists of a forward primer having the sequence CCT TTC TGC TCT TCC AAC GCC GAC GGG GCT (SEQ ID NO: 1), and a reverse primer having the sequence ACT TTC AAC CTG CAG TTA CCC AGC GGC CCC (SEQ ID NO: 2).

11. The method according to claim 1 for constructing a library of hydrophilic peptides, wherein the nucleic acid molecule provided further comprises codons for at least one hydrophilic amino acid to be added into the peptide of interest.

12. The method according to claim 11, wherein the peptide GRENYHGCTTHWGFTLC (SEQ ID NO: 24) is produced.

13. The method according to claim 1 for constructing a library of hydrophilic peptides, wherein the nucleic acid molecule provided further comprises codons for at least one hydrophilic amino acid for replacing an amino acid non-critical for the activity of the peptide of interest.

14. The method according to claim 1 for producing a pool of peptides, wherein the nucleic acid molecule provided comprises a plurality of nucleotide sequences encoding peptides of interest.

15. The method according to claim 14, comprising a flurther step of screening the peptide pool obtained for improved solubility properties.

16. The method according to claim 1 for producing a peptide with an unnatural amino acid, wherein the method further comprises the steps of
providing a host cell auxotrophic for a naturally occurring amino acid to be replaced with said unnatural amino acid,
expressing the peptide-intein-fusion in said auxotrophic host cell in the presence of an amino acid analogue.

17. The method according to claim 16, wherein the peptide CTTH(5-fluoro-W)GFTLC (SEO ID NO: 20) is produced.

18. The method according to claim 16, wherein the peptide CTTH(6-fluoro-W)GFTLC (SEQ ID NO: 20) is produced.

19. The peptide CTTH(5-fluoro-W)GFTLC (SEQ ID NO: 20) having improved serum stability.

20. The peptide GRENYHGCTTHWGFTLC (SEQ ID NO: 24) having improved solubility in water.

21. The peptide CTTH(5-fluoro-W)GFTLC (SEQ ID NO: 20), which is obtainable according to claim 16.

22. The peptide GRENYHGCTTHWGFTLC (SEQ ID NO: 24), which is obtainable according to claim 11.

23. A method for producing a peptide with an unnatural amino acid, wherein the method comprises the steps of
expressing a library of peptides containing an amino acid analogue on a phage using an auxotrophic host,
selecting a peptide of interest containing an amino acid analogue using phage display in an auxotrophic host,
transferring the nucleic acid encoding said peptide into an intein vector, and
expressing the peptide of interest according to the method of claim 16.


SEQUENCE LISTING (27 sequences)

SEQ ID NO: 1 - DNA, 30 bp, Artificial Sequence
  Synthetic intein Fwd SapI primer
cctttctgctcttccaacgccgacggggct

SEQ ID NO: 2 - DNA, 30 bp, Artificial Sequence
  Synthetic intein Rev PstI primer
actttcaacctgcagttacccagcggcccc

SEQ ID NO: 3 - DNA, 59 bp, Artificial Sequence
  Synthetic oligonucleotide encoding SEQ ID NO 10
ggtggtgctcttccaactgtacgacccattggggatttactttatgttaactgcaggcg

SEQ ID NO: 4 - DNA, 15 bp, Artificial Sequence
  Synthetic primer used to convert SEQ ID NO 3 to double-stranded form
cgcctgcagttaaca

SEQ ID NO: 5 - DNA, 75 bp, Artificial Sequence
  Synthetic degenerate oligonucleotide used in the cloning of the intein-peptide fusions
ggtggttgctcttccaacggccgccvavvavtatvavggctgtaccacccatttactttatgttaactgcaggcg

SEQ ID NO: 6 - DNA, 17 bp, Artificial Sequence
  Transposon specific primer
atcagcggccgcgatcc

SEQ ID NO: 7 - DNA, 21 bp, Artificial Sequence
  Transposon specific primer
ttattcggtcgaaaaggatcc

SEQ ID NO: 8 - protein, 4 aa, Unknown
  Peptide derived from phage
ADGA

SEQ ID NO: 9 - protein, 4 aa, Unknown
  Peptide derived from phage
GAAG

SEQ ID NO: 10 - protein, 10 aa, Unknown
  Recombinant peptide prepared by the intein system
CTTHWGFTLC

SEQ ID NO: 11 - protein, 10 aa, Unknown
  Non-cyclic synthetic control peptide
STTHWGFTLS

SEQ ID NO: 12 - protein, 10 aa, Artificial Sequence
  Description of Artificial Sequence Ala-substitution of the CTT-peptide
CATHWGFTLC

SEQ ID NO: 13 - protein, 10 aa, Artificial Sequence
  Description of Artificial Sequence Ala-substitution of the CTT-peptide
CTAHWGFTLC

SEQ ID NO: 14 - protein, 10 aa, Artificial Sequence
  Description of Artificial Sequence Ala-substitution of the CTT-peptide
CTTAWGFTLC

SEQ ID NO: 15 - protein, 10 aa, Artificial Sequence
  Description of Artificial Sequence Ala-substitution of the CTT-peptide
CTTHAGFTLC

SEQ ID NO: 16 - protein, 10 aa, Artificial Sequence
  Description of Artificial Sequence Ala-substitution of the CTT-peptide
CTTHWAFTLC

SEQ ID NO: 17 - protein, 10 aa, Artificial Sequence
  Description of Artificial Sequence Ala-substitution of the CTT-peptide
CTTHWGATLC

SEQ ID NO: 18 - protein, 9 aa, Artificial Sequence
  Description of Artificial Sequence Ala-substitution of the CTT-peptide
CTTHWGFAL

SEQ ID NO: 19 - protein, 10 aa, Artificial Sequence
  Description of Artificial Sequence Ala-substitution of the CTT-peptide
CTTHWGFALC

SEQ ID NO: 20 - protein, 10 aa, Artificial Sequence
  Description of Artificial Sequence CTT-peptide with a tryptophan analogue at position 5
CTTHXGFTLC

SEQ ID NO: 21 - protein, 10 aa, Artificial Sequence
  Description of Artificial Sequence Control sequence
CERGGLETSC

SEQ ID NO: 22 - protein, 9 aa, Artificial Sequence
  Description of Artificial Sequence Control sequence
CPCFLLGCC

SEQ ID NO: 23 - protein, 17 aa, Artificial Sequence
  Description of Artificial Sequence CTT-peptide with additional hydrophilic amino acids
GRXXYXGCTTHWGFTLC

SEQ ID NO: 24 - protein, 17 aa, Artificial Sequence
  Description of Artificial Sequence CTT-peptide with additional hydrophilic amino acids
GRENYHGCTTHWGFTLC

SEQ ID NO: 25 - protein, 9 aa, Artificial Sequence
  Description of Artificial Sequence Synthetic peptide derived from phage
ADGAXGAAG

SEQ ID NO: 26 - protein, 8 aa, Artificial Sequence
  Description of Artificial Sequence MMP-2 specific fluorescent peptide substrate
XPLAXXAR

SEQ ID NO: 27 - protein, 10 aa, Artificial Sequence
  Description of Artificial Sequence recombinant peptide prepared by the intein system
CTTHWGFTAC